(19) United States
(12) Patent Application Publication
(10) Pub. No.: US 2022/0276231 A1
(43) Pub. Date: Sep. 1, 2022

(54) METHOD FOR MEASURING BETA-1,3-1,6-GLUCAN

(71) Applicants: OLYMPUS CORPORATION, Tokyo (JP); Tokyo University of Pharmacy & Life Sciences, Tokyo (JP)

(72) Inventors: Hidetaka NAKATA, Hachioji-shi (JP); Yoshiyuki ADACHI, Tokyo (JP); Kenichi ISHIBASHI, Tokyo (JP); Daisuke YAMANAKA, Tokyo (JP); Naohito OHNO, Tokyo (JP)

(73) Assignees: OLYMPUS CORPORATION, Tokyo (JP); Tokyo University of Pharmacy & Life Sciences, Tokyo (JP)

(21) Appl. No.: 17/747,590

(22) Filed: May 18, 2022

Related U.S. Application Data
(63) Continuation of application No. PCT/JP2020/043393, filed on Nov. 20, 2020.

(30) Foreign Application Priority Data
Nov. 20, 2019 (JP) 2019-209679

(51) Int. Cl. G01N 33/53; G01N 33/543; G01N 33/58

(52) U.S. Cl. CPC G01N 33/5308; G01N 33/54326; G01N 33/582; G01N 2400/24; G01N 2800/26

(57) ABSTRACT

The present invention provides a method for quantitatively detecting β-1,3-1,6-glucan separately from β-1,3-glucan and β-1,3-1,4-glucan. The present invention is a method for measuring β-1,3-1,6-glucan, the method including: a step for mixing β-glucan in a test sample, a molecule that specifically binds to a β-(1→3) bond, and a molecule that specifically binds to a β-(1→6) bond to form a complex containing the molecule that specifically binds to a β-(1→3) bond and the molecule that specifically binds to a β-(1→6) bond; a step for detecting the complex; and a step for measuring the amount of β-1,3-1,6-glucan in the test sample, on the basis of the results of the detection.

This application is a continuation application of PCT/JP2020/043393, filed Nov. 20, 2020, which claims priority to Japanese Patent Application No. 2019-209679, filed Nov. 20, 2019, the contents of each of which are incorporated herein by reference.

### TECHNICAL FIELD

The present invention relates to a method for measuring β-1,3-1,6-glucan containing a β-(1→3) bond and a β-(1→6) bond.

### INCORPORATION BY REFERENCE OF SEQUENCE LISTING

The Sequence Listing in an ASCII text file, named 40937Z_SequenceListing.txt of 8 KB, created on May 18, 2022, and submitted to the United States Patent and Trademark Office via EFS-Web, is incorporated herein by reference.

### BACKGROUND ART

Among glucans that are polysaccharides where glucose is linked by a glycosidic bond, β-glucan is a general term for polymers linked by a β-glycosidic bond. Although β-glucan is present in fungi, bacteria, plants and the like, it is absent in humans. The β-glycosidic bond is mainly composed of β-(1→3) bonds, β-(1→4) bonds, and β-(1→6) bonds. The β-glucan contained in fungi and bacteria mainly contains β-(1→3) bonds and β-(1→6) bonds, and the β-glucan contained in plants mainly contains β-(1→3) bonds and β-(1→4) bonds.

By utilizing the fact that humans do not contain β-glucan and by detecting β-glucan contained in a sample collected from a human, it is possible to detect fungi, bacteria and the like contained in the sample. In particular, detection of β-glucan has been used for deep mycosis tests. Deep mycosis is a disease in which a fungal infection extends to internal organs and often occurs in immunosuppressed patients. Antifungal drugs are usually given to patients who are diagnosed as having pathogenic fungi present in the body causing deep mycosis such as Aspergillus and Candida by the deep mycosis test.

For the deep mycosis test, the Limulus reaction using Limulus factor G, which is a β-1,3-glucan (β-glucan composed of β-(1→3) bonds) responsive protein derived from a horseshoe crab, is currently being used. Further, Patent Document 1 discloses a method for quantifying β-1,3-1,6-glucan (β-glucan composed of β-(1→3) bonds and β-(1→6) bonds) by mixing a sample to be measured with an extracellular enzyme solution derived from a fungus producing β-1,3-glucanase and an extracellular enzyme solution derived from a fungus producing β-1,6-glucanase, and measuring the amount of glucose produced by decomposition.

### CITATION LIST

### Patent Documents

[Patent Document 1] Japanese Unexamined Patent Application, First Publication No. 2010-41957

[Patent Document 2] International Patent Publication No. 2018/212095

### SUMMARY OF INVENTION

### Technical Problem

In the Limulus reaction using factor G that recognizes β-1,3-glucan, it is not possible to distinguish between the β-glucan derived from a fungus and the β-glucan derived from a plant. Further, in the method described in Patent Document 1, in addition to the glucan containing both β-1,3-glucan and β-1,6-glucan, β-1,3-glucan composed of only β-(1→3) bonds and β-glucan containing both β-1,3-glucan and β-1,4-glucan (β-1,3-1,4-glucan) are also detected. That is, with these methods, it is not possible to detect the fungal-derived β-glucan and the plant-derived β-glucan containing a β-(1→3) bond while distinguishing them from each other.

In particular, in the deep mycosis test, it is important to distinguish between the fungal-derived β-glucan and the plant-derived β-glucan. For example, there are cases where the plant-derived β-glucan is introduced into the human body by using gauze in a surgical procedure, administering a formulation using a cellulosic filter medium in the formulation process, or hemodialysis using a cellulosic dialysis membrane. As described above, if the fungal-derived β-glucan and the plant-derived β-glucan cannot be distinguished in the deep mycosis test for samples collected from humans in which the plant-derived β-glucan has been introduced into the body, samples contaminated with the plant-derived β-glucan may become false positives, resulting in erroneous diagnosis of deep mycosis and unnecessary administration of antifungal drugs.

The present invention has an object of providing a method for quantitatively detecting β-1,3-1,6-glucan separately from β-1,3-glucan and β-1,3-1,4-glucan.

### Solution to Problem

As a result of intensive research in order to solve the above problems, the inventors of the present invention have found that by combining a molecule that specifically binds to a β-(1→3) bond and a molecule that specifically binds to a β-(1→6) bond and detecting a molecule that binds to both of these molecules, β-1,3-1,6-glucan can be specifically detected without detecting a molecule that has a β-(1→3) bond but has no β-(1→6) bond, or a molecule that has a β-(1→6) bond but has no β-(1→3) bond, thereby completing the present invention.

That is, a method for measuring β-1,3-1,6-glucan, a method for evaluating a likelihood of fungal infection, and a kit for measuring β-1,3-1,6-glucan according to the present invention include the following aspects [1] to [13].

[1] A method for measuring β-1,3-1,6-glucan, the method including: a step for mixing β-glucan in a test sample, a molecule that specifically binds to a β-(1→3) bond, and a molecule that specifically binds to a β-(1→6) bond to form a complex containing the aforementioned molecule that specifically binds to a β-(1→3) bond and the aforementioned molecule that specifically binds to a β-(1→6) bond;

a step for detecting the aforementioned complex separately from a β-glucan bonded to only one of said molecule that specifically binds to a β-(1→3) bond and said molecule that specifically binds to a β-(1→6); and

a step for measuring an amount of β-1,3-1,6-glucan in the aforementioned test sample based on a result of the aforementioned detection.

[2] The method for measuring β-1,3-1,6-glucan according to the above [1], wherein the aforementioned molecule that specifically binds to a β-(1→6) bond is at least one selected from the group consisting of an enzyme-inactivated mutant of β-1,6-glucanase and an anti-β-1,6-glucan antibody.

[3] The method for measuring β-1,3-1,6-glucan according to the above [1] or [2], wherein the aforementioned molecule that specifically binds to a β-(1→3) bond is at least one selected from the group consisting of a horseshoe crab-derived factor G or a mutant thereof, a protein containing a carbohydrate recognition domain of dectin-1 or a mutant thereof, a β-glucan recognition protein or a mutant thereof, an enzyme-inactivated mutant of β-1,3-glucanase and an anti-β-1,3-glucan antibody.

[4] The method for measuring β-1,3-1,6-glucan according to any one of the above [1] to [3], wherein at least one of the aforementioned molecule that specifically binds to a β-(1→3) bond and the aforementioned molecule that specifically binds to a 13-(1→6) bond is removed from the aforementioned complex, prior to the step for detecting the aforementioned complex.

[5] The method for measuring β-1,3-1,6-glucan according to any one of the above [1] to [4], wherein at least one of the aforementioned molecule that specifically binds to a β-(1→3) bond and the aforementioned molecule that specifically binds to a β-(1→6) bond is labeled with a labeling material, and detection of the aforementioned complex is carried out by detecting a signal emitted from the aforementioned labeling material.

[6] The method for measuring β-1,3-1,6-glucan according to the above [5], wherein one of the aforementioned molecule that specifically binds to a β-(1→3) bond and the aforementioned molecule that specifically binds to a β-(1→6) bond is labeled with the aforementioned labeling material, while the other is immobilized on a solid phase support.

[7] The method for measuring β-1,3-1,6-glucan according to the above [6], wherein the aforementioned solid phase support is a magnetic bead.

[8] The method for measuring β-1,3-1,6-glucan according to the above [6] or [7], wherein the aforementioned solid phase support is modified with a biotin-binding molecule, and among the aforementioned molecule that specifically binds to a β-(1→3) bond and the aforementioned molecule that specifically binds to a β-(1→6) bond, the molecule immobilized on the aforementioned solid phase support is a biotin-modified molecule.

[9] The method for measuring β-1,3-1,6-glucan according to any one of the above [5] to [8], wherein the aforementioned labeling material is a luminescent material.

[10] The method for measuring β-1,3-1,6-glucan according to the above [9], wherein the aforementioned labeling material is a fluorescent material.

[11] The method for measuring β-1,3-1,6-glucan according to any one of the above [1] to [10], wherein the aforementioned complex is detected by a scanning single-molecule counting method.

[12] A method for evaluating a likelihood of fungal infection, the method including: a step of performing the method for measuring β-1,3-1,6-glucan according to any one of the above [1] to [11] using a biological sample collected from a test animal as a test sample, and measuring an amount of β-1,3-1,6-glucan in the aforementioned test sample; and

a step of evaluating a likelihood of infection of the aforementioned test animal with a fungus based on an amount of β-1,3-1,6-glucan in the aforementioned test sample obtained by the aforementioned measurement.

[13] A kit for measuring β-1,3-1,6-glucan, the kit including a molecule that specifically binds to a β-(1→3) bond and a molecule that specifically binds to a β-(1→6) bond, wherein

either one of said molecules is bindable to a solid phase support, and the other one binds to a labeling material which is detectable.

### Advantageous Effects of Invention

The method for measuring β-1,3-1,6-glucan according to the present invention can measure β-1,3-1,6-glucan separately from β-1,3-glucan or β-1,3-1,4-glucan. For this reason, this method can accurately quantify the amount of β-1,3-1,6-glucan in a sample that may contain β-1,3-glucan or β-1,3-1,4-glucan, and in particular, can be suitably used for evaluating the likelihood of infection of humans with a fungus, which may contain β-glucan derived from a plant as a contaminant.

Further, by using the kit for measuring β-1,3-1,6-glucan according to the present invention, this method for measuring β-1,3-1,6-glucan can be performed more easily.

### BRIEF DESCRIPTION OF DRAWINGS

FIG. 1 is a diagram showing the results of detection of each concentration of β-glucan by a scanning single-molecule counting method using fluorescently modified S-BGRP and a biotin-modified β-1,6-glucanase E321 mutant in Example 1. FIG. 1 (A) is a result using CSBG, FIG. 1 (B) is a result using ASBG, and FIG. 1 (C) is a result using Pollen BG.

FIG. 2 is a diagram showing the results of detection of each concentration of β-glucan by fluorescence intensity measurement using fluorescently modified S-BGRP and a biotin-modified β-1,6-glucanase E321 mutant in Example 2. FIG. 2 (A) is a result using CSBG, FIG. 2 (B) is a result using ASBG, and FIG. 2 (C) is a result using Pollen BG.

FIG. 3 is a diagram showing the results of detection of CSBG added to human serum by a scanning single-molecule counting method using fluorescently modified S-BGRP and a biotin-modified β-1,6-glucanase E321 mutant, and measurement of the additional recovery rate of CSBG (%: ([number of peaks of human serum-added sample]/[number of peaks of human serum-free sample]×100) in Example 5. FIG. 3 (A) is a result using serum A, FIG. 3 (B) is a result using serum B, and FIG. 3 (C) is a result using serum C.

FIG. 4 is a diagram showing the results of detection of CSBG by a scanning single-molecule counting method using fluorescently modified BmBGRP and a biotin-modified β-1,6-glucanase E321 mutant in Example 6.

FIG. 5 is a diagram showing the results of detection of CSBG by a scanning single-molecule counting method using fluorescently modified S-BGRP and a biotin-modified anti-β-1,6 glucan antibody in Example 7.

### DESCRIPTION OF EMBODIMENTS

In the present invention and the present specification, the term “β-1,3-1,6-glucan” means a β-glucan containing a β-(1→3) bond and a β-(1→6) bond. The β-1,3-1,6-glucan may be a β-glucan composed of only β-(1→3) bonds and β-(1→6) bonds, or may be a β-glucan containing, in addition to both of these bonds, other β-glucosidic bonds such as β-(1→4) bonds.

The method for measuring β-1,3-1,6-glucan according to the present invention is characterized by allowing β-1,3-1,6-glucan to bind to both of a molecule that specifically binds to a β-(1→3) bond (hereinafter, may be referred to as “β-1,3 glucan-binding molecule”) and a molecule that specifically binds to a β-(1→6) bond (hereinafter, may be referred to as “β-1,6 glucan-binding molecule”), and detecting a formed tripartite complex. By allowing to bind to both of the β-1,3 glucan-binding molecule and the β-1,6 glucan-binding molecule to form a complex, β-1,3-1,6-glucan can be specifically detected by distinguishing it from a molecule that specifically binds to only a β-(1→3) bond or only a β-(1→6) bond, such as, β-1,3-glucan, β-1,3-1,4-glucan or the like.

The β-1,3 glucan-binding molecule used in the present invention is not particularly limited as long as it can bind to a β-(1→3) bond and does not bind to other β-glucosidic bonds. Examples of the β-1,3 glucan-binding molecule include a horseshoe crab-derived factor G or a mutant thereof, a protein containing a carbohydrate recognition domain of dectin-1 or a mutant thereof, a β-glucan recognition protein (BGRP) or a mutant thereof, an enzyme-inactivated mutant of β-1,3-glucanase and an anti-β-1,3-glucan antibody. These proteins may be extracted and purified from animals or microorganisms, or may be recombinant proteins. The recombinant protein can be synthesized by a conventional method based on the amino acid sequence information. The β-1,3 glucan-binding molecule used in the present invention may be one type or two or more types.

The horseshoe crab-derived factor G used in the present invention may be a protein having the same amino acid sequence as that of a factor G purified from the wild horseshoe crab blood cell extract (wild-type factor G), or may be a mutant obtained by introducing various mutations into the wild-type factor G (mutant factor G) as long as the specific binding ability with respect to the β-(1→3) bond is not impaired. Examples of horseshoe crabs include Tachypleus tridentatus, Tachypleus gigas, Limulus polyphemus and Carcinoscorpius rotundicauda.

Dectin-1 is a membrane protein that belongs to C-type lectin expressed in dendritic cells and macrophages and recognizes a β-glucan containing a β-(1→3) bond. The dectin-1 used in the present invention is preferably a human-derived dectin-1, but may be a dectin-1 derived from a biological species other than humans. Further, the carbohydrate recognition domain-containing protein of dectin-1 may be any protein containing the carbohydrate recognition domain of dectin-1, and may be a partial protein of dectin-1 composed only of the carbohydrate recognition domain, a partial protein of dectin-1 outside the cell membrane, or a full-length protein of dectin-1. Moreover, the β-1,3 glucan-binding molecule used in the present invention may be a mutant (mutant dectin-1) obtained by introducing various mutations into the protein containing the carbohydrate recognition domain of dectin-1 as long as the specific binding ability with respect to the β-(1→3) bond is not impaired.

The BGRP used in the present invention may be any BGRP that can bind to β-(1→3) bonds and does not bind to other β-glucosidic bonds. The BGRP may be a wild-type BGRP derived from any biological species, or may be a mutant (mutant BGRP) obtained by introducing various mutations into the wild-type BGRP as long as the specific binding ability to the β-(1→3) bond is not impaired. Examples of the BGRP used in the present invention include S-BGRP (SEQ ID NO: 1), BmBGRP (derived from Bombyx mori) (SEQ ID NO: 3), PiBGRP (derived from Plodia interpunctera), TcBGRP (derived from Tribolium castaneum), and TmBGRP (derived from Tenebrio molita).

The enzyme-inactivated mutant of β-1,3-glucanase is a mutant in which the enzyme activity of β-1,3-glucanase (EC 3.2.1.39) is eliminated or reduced, and is a mutant obtained by introducing a mutation that eliminates or reduces the enzyme activity while retaining the binding ability with respect to the β-(1→3) bond. The enzyme-inactivated mutant of β-1,3-glucanase used in the present invention may be a mutant obtained by introducing a necessary mutation into a β-1,3-glucanase derived from any biological species. Examples of the mutant into which the mutation that eliminates or reduces the enzyme activity has been introduced include a mutant into which a mutation has been introduced into an amino acid essential for the enzyme activity in the enzyme active site, and a mutant in which the enzyme active site has been deleted. Further, the enzyme-inactivated mutant of β-1,3-glucanase used in the present invention may be a mutant into which various mutations have been introduced within a site other than the enzyme active site of β-1,3-glucanase, in addition to a mutation that eliminates or reduces the enzyme activity of β-1,3-glucanase, as long as the specific binding ability with respect to the β-(1→3) bond is not impaired.

The anti-β-1,3-glucan antibody used in the present invention may be any antibody that can bind to β-(1→3) bonds and does not bind to other β-glucosidic bonds. This anti-β-1,3-glucan antibody may be any class of antibody, and may be an IgG antibody, or an IgM antibody. Further, the antibody may be derived from any biological species, and may be any of a monoclonal antibody, a polyclonal antibody, a chimeric antibody, and a humanized antibody. Moreover, it may be a low molecular weight antibody such as a Fab antibody or a scFv antibody.

The β-1,6 glucan-binding molecule used in the present invention is not particularly limited as long as it can bind to a β-(1→6) bond and does not bind to other β-glucosidic bonds. Examples of the β-1,6 glucan-binding molecule include an enzyme-inactivated mutant of β-1,6-glucanase and an anti-β-1,6-glucan antibody. These proteins may be extracted and purified from animals or microorganisms, or may be recombinant proteins. The recombinant protein can be synthesized by a conventional method based on the amino acid sequence information. The β-1,6 glucan-binding molecule used in the present invention may be one type or two or more types.

The enzyme-inactivated mutant of β-1,6-glucanase is a mutant in which the enzyme activity of β-1,6-glucanase (EC 3.2.1.75) is eliminated or reduced, and is a mutant obtained by introducing a mutation that eliminates or reduces the enzyme activity while retaining the binding ability with respect to the β-(1→6) bond. The enzyme-inactivated mutant of β-1,6-glucanase used in the present invention may be a mutant obtained by introducing a necessary mutation into a β-1,6-glucanase derived from any biological species. Examples of the mutant into which the mutation that eliminates or reduces the enzyme activity has been introduced include a mutant into which a mutation has been introduced into an amino acid essential for the enzyme activity in the enzyme active site, and a mutant in which the enzyme active site has been deleted. Alternatively, the enzyme-inactivated mutant of β-1,6-glucanase used in the present invention may be a mutant into which various mutations have been introduced within a site other than the enzyme active site of β-1,6-glucanase, in addition to a mutation that eliminates or reduces the enzyme activity of β-1,6-glucanase, as long as the specific binding ability with respect to the β-(1→6) bond is not impaired.

Examples of the enzyme-inactivated mutant of β-1,6-glucanase used in the present invention include a mutant of β-1,6-glucanase, which is a mutant (β-1,6-glucanase E321 mutant) in which Glu (E) corresponding to the 321st Glu (E) of an amino acid sequence represented by SEQ ID NO: 2 (amino acid sequence of β-1,6-glucanase derived from red bread mold (Neurospora crassa)) is substituted with an amino acid residue selected from the group consisting of Gln (Q), Gly (G), Ala (A), Leu (L), Tyr (Y), Met (M), Ser (S), Asn (N) and His (H), and a mutant of β-1,6-glucanase (EC 3.2.1.75), which is a mutant (β-1,6-glucanase E225/E321 mutant) (Patent Document 2) in which Glu (E) corresponding to the 225th and 321st Glu (E) of the amino acid sequence represented by SEQ ID NO: 2 is substituted with an amino acid residue selected from the group consisting of Gln (Q), Gly (G), Ala (A), Leu (L), Tyr (Y), Met (M), Ser (S), Asn (N) and His (H). Alternatively, it may be a mutant into which various mutations have been introduced, within a site other than the enzyme active site of β-1,6-glucanase, to the β-1,6-glucanase E321 mutant or the β-1,6-glucanase E225/E321 mutant as long as the specific binding ability with respect to the β-(1→6) bond is not impaired.

The anti-β-1,6-glucan antibody used in the present invention may be any antibody that can bind to β-(1→6) bonds and does not bind to other β-glucosidic bonds. This anti-β-1,6-glucan antibody may be any class of antibody, and may be an IgG antibody, or an IgM antibody. Further, the antibody may be derived from any biological species, and may be any of a monoclonal antibody, a polyclonal antibody, a chimeric antibody, and a humanized antibody. Moreover, it may be a low molecular weight antibody such as a Fab antibody or a scFv antibody.

It should be noted that in the present invention and the present specification, examples of mutations introduced into proteins include, in addition to those specifically described, mutations caused by deletions, insertions, substitutions or additions of one or several (preferably 10 or less, more preferably 7 or less, and most preferably 5 or less) amino acids. Further, the sequence identity between the amino acid sequence before the introduction of the mutation and the amino acid sequence after the introduction of the mutation is preferably 70% or more, more preferably 80% or more, still more preferably 90% or more, and most preferably 95% or more.

It should be noted that the sequence identity (homology) between the amino acid sequences is determined as a ratio of matching amino acids with respect to the entire amino acid sequence excluding the gap within an alignment obtained by juxtaposing the two amino acid sequences while interposing a gap in parts corresponding to the insertion and deletion so that the corresponding amino acids match most. The sequence identity between amino acid sequences can be determined by using various homology search software known in the art.

The β-1,3 glucan-binding molecule used in the present invention may have other peptides or proteins fused to the N-terminal or C-terminal as long as the specific binding ability with respect to the β-(1→3) bond is not impaired. Similarly, the β-1,6 glucan-binding molecule used in the present invention may have other peptides or proteins fused to the N-terminal or C-terminal as long as the specific binding ability with respect to the β-(1→6) bond is not impaired. Examples of these peptides and the like include tags that are widely used in the expression and purification of recombinant proteins such as histidine tags, hemagglutinin (HA) tags, Myc tags, and Flag tags.

As a method for measuring β-1,3-1,6-glucan according to the present invention, from the viewpoint of obtaining higher detection sensitivity, it is preferable to use BGRP as the β-1,3 glucan-binding molecule and the enzyme-inactivated mutant of β-1,6-glucanase as the β-1,6 glucan-binding molecule; it is more preferable to use BGRP as the β-1,3 glucan-binding molecule, and the β-1,6-glucanase E321 mutant or the β-1,6-glucanase E225/E321 mutant as the β-1,6 glucan-binding molecule; and it is still more preferable to use S-BGRP, BmBGRP, PiBGRP, TcBGRP, or TmBGRP as the β-1,3 glucan-binding molecule, and the β-1,6-glucanase E321 mutant or the β-1,6-glucanase E225/E321 mutant as the β-1,6 glucan-binding molecule.

More specifically, the method for measuring β-1,3-1,6-glucan according to the present invention includes: a step of mixing β-glucan in a test sample, a molecule that specifically binds to a β-(1→3) bond, and a molecule that specifically binds to a β-(1→6) bond, thereby forming a complex containing the aforementioned molecule that specifically binds to a β-(1→3) bond and the aforementioned molecule that specifically binds to a β-(1→6) bond (complex formation step); a step of detecting the aforementioned complex (detection step); and a step of measuring the amount of β-1,3-1,6-glucan in the aforementioned test sample based on a result of the aforementioned detection (quantification step).

The test sample subjected to the method for measuring β-1,3-1,6-glucan according to the present invention is not particularly limited as long as it is a sample expected to contain β-1,3-1,6-glucan or a sample necessary to determine whether or not β-1,3-1,6-glucan is contained. Examples of the sample include a biological sample, and a fraction containing β-glucan obtained by extraction/purification or the like from a biological sample. Further, the test sample may be subjected to the addition of a surfactant, treatment with various enzymes, dilution, heating and the like, before being subjected to the method for measuring β-1,3-1,6-glucan according to the present invention, as long as the β-1,3-1,6-glucan contained in the sample is not decomposed.

The biological sample is a sample collected from a living organism, and examples thereof include a piece of tissue, body fluids such as blood, lymph, bone marrow aspirate, ascitic fluid, exudate, amniotic fluid, sputum, saliva, semen, bile, pancreatic fluid and urine; feces, intestinal lavage fluid, lung lavage fluid, bronchial lavage fluid and bladder lavage fluid, collected from living bodies. It should be noted that the method for collecting a piece of tissue from the living body is not particularly limited, and examples thereof include a blood sample, a serum sample, a plasma sample, a biopsy sample collected by needle puncture or endoscopy, and a surgical sample.

In the method for measuring β-1,3-1,6-glucan according to the present invention, the order of mixing β-glucan in the test sample, the β-1,3 glucan-binding molecule and the β-1,6 glucan-binding molecule is not particularly limited. Further, when mixing these three components, water or a buffer may be used as a solvent, if necessary. Examples of the buffer include a phosphate buffer such as phosphate buffered saline (PBS, pH 7.4), a Tris buffer and a HEPES buffer.

For example, it is possible to mix either one of the β-1,3 glucan-binding molecule and the β-1,6 glucan-binding molecule with the test sample diluted with a buffer or the like if necessary, and after incubating for a predetermined time as needed, add the other remaining component to the obtained mixture, followed by incubation and mixing for a predetermined time if required; or it is possible to mix the β-1,3 glucan-binding molecule and the β-1,6 glucan-binding molecule in a buffer or the like in advance, and mix the obtained mixture with the test sample. Each incubation can be carried out, for example, at room temperature (1 to 30° C.) to 37° C. for about 1 minute to 2 hours.

When the test sample, the β-1,3 glucan-binding molecule and the β-1,6 glucan-binding molecule are mixed, in the β-glucan in the test sample, the β-(1→3) bond and the β-1,3 glucan-binding molecule bind, and the β-(1→6) bond and the β-1,6 glucan-binding molecule bind. Both the β-1,3 glucan-binding molecule and the β-1,6 glucan-binding molecule bind to the β-1,3-1,6-glucan in the test sample to form a complex. In other words, by detecting a complex containing both the β-1,3 glucan-binding molecule and the β-1,6 glucan-binding molecule in one molecule, β-1,3-1,6-glucan in the test sample can be detected.

The method for detecting a complex containing both the β-1,3 glucan-binding molecule and the β-1,6 glucan-binding molecule is not particularly limited. For example, at least one of the β-1,3 glucan-binding molecule and the β-1,6 glucan-binding molecule is labeled with a labeling material in advance. By detecting a signal emitted from this labeling material, the complex containing both the β-1,3 glucan-binding molecule and the β-1,6 glucan-binding molecule can be detected.

As the amount of β-1,3-1,6-glucan contained in the test sample increases, the amount of the complex containing both the β-1,3 glucan-binding molecule and the β-1,6 glucan-binding molecule increases, and the amount of the labeling material contained in the complex also increases. That is, the amount of β-1,3-1,6-glucan in the test sample can be quantified, based on the intensity of the signal of the labeling material emitted from the complex containing both the β-1,3 glucan-binding molecule and the β-1,6 glucan-binding molecule, and the amount of particles emitting this signal.

The labeling material is preferably a luminescent material because it has excellent sensitivity. The luminescent material means a material that emits light by fluorescence, phosphorescence, chemiluminescence, bioluminescence, light scattering, or the like. Examples of the labeling material other than the luminescent material include radioactive isotopes.

In particular, since the fluorescence signal can be detected with high sensitivity and measurement at a single molecule level is relatively easy, the labeling material is preferably a fluorescent material that labels a β-1,3 glucan-binding molecule or a β-1,6 glucan-binding molecule. The fluorescent material is not particularly limited as long as it is a material that emits fluorescence by irradiation of light of a specific wavelength, and can be appropriately selected and used from among fluorescent materials usually used for labeling proteins, nucleic acids, low molecular weight compounds or the like, quantum dots, and the like. More specifically, examples of the fluorescent material include fluorescein isothiocyanate (FITC), fluorescein, rhodamine, TAMRA, NBD, tetramethylrhodamine (TMR), Cy5 (manufactured by GE Healthcare Bioscience), Alexa Fluor (registered trademark) series (manufactured by Invitrogen) and ATTO dye series (manufactured by ATTO-TEC GmbH). Examples of quantum dots include CdSe and the like.

When both the β-1,3 glucan-binding molecule and the β-1,6 glucan-binding molecule are labeled with fluorescent materials having different fluorescence characteristics, particles emitting two types of fluorescence having different wavelengths emitted from both fluorescent materials can be detected as a complex containing both the β-1,3 glucan-binding molecule and the β-1,6 glucan-binding molecule. It should be noted that the expression “different fluorescence characteristics” means that the wavelengths of fluorescence emitted by the irradiation of excitation light are so different that they can be detected separately. Further, when labeling either one of the β-1,3 glucan-binding molecule and the β-1,6 glucan-binding molecule with a fluorescent material to serve as a donor and the other with a quenching material to serve as an acceptor, by detecting the fluorescence emitted by fluorescence resonance energy transfer (FRET) as an indicator, it can be detected as a complex containing both the β-1,3 glucan-binding molecule and the β-1,6 glucan-binding molecule. The fluorescent material to serve as a donor and the quenching material to serve as an acceptor are not particularly limited as long as they are a combination that produces FRET, and can be appropriately selected and used from among those commonly used.

It is also possible to label either one of the β-1,3 glucan-binding molecule and the β-1,6 glucan-binding molecule with a labeling material and to immobilize the other on a solid phase support. In this case, the complex containing both the β-1,3 glucan-binding molecule and the β-1,6 glucan-binding molecule is also immobilized on the solid phase support. Accordingly, the complex containing both the β-1,3 glucan-binding molecule and the β-1,6 glucan-binding molecule can be detected in a state where the free labeling material is removed by making use of a solid-liquid separation treatment using a solid phase support. For example, when the β-1,3 glucan-binding molecule is labeled with a labeling material and the β-1,6 glucan-binding molecule is immobilized on a solid phase support, by performing a solid-liquid separation treatment after forming the complex, the β-1,3 glucan-binding molecule that is not bound to β-1,3-1,6-glucan is removed from the complex immobilized on the solid phase support. Similarly, when the β-1,6 glucan-binding molecule is labeled with a labeling material and the β-1,3 glucan-binding molecule is immobilized on a solid phase support, by performing a solid-liquid separation treatment after forming the complex, the β-1,6 glucan-binding molecule that is not bound to β-1,3-1,6-glucan is removed from the complex immobilized on the solid phase support.

The β-1,3 glucan-binding molecule or the β-1,6 glucan-binding molecule may be immobilized by directly binding to the solid phase support, or may be modified with a linker material capable of binding to the solid phase support. In the latter case, the complex containing both the β-1,3 glucan-binding molecule and the β-1,6 glucan-binding molecule is immobilized on the solid phase support by this linker material.

The shape, material and the like of the solid phase support are not particularly limited as long as it is a solid provided with a site that directly or indirectly binds to the linker material. For example, it may be particles such as beads that can be suspended in water and can be separated from a liquid by a general solid-liquid separation treatment, may be a membrane, or may be a container, a chip substrate or the like. Specific examples of the solid phase support include magnetic beads, silica beads, agarose gel beads, polyacrylamide resin beads, latex beads, polystyrene beads and other plastic beads, ceramic beads, zirconia beads, silica membranes, silica filters and plastic plates.

Examples of the linker material include biotin, avidin, streptavidin, glutathione, dinitrophenol (DNP), digoxigenin, digoxin, sugar chains composed of two or more sugars, polypeptides composed of four or more amino acids such as a His tag, a Flag tag, and a Myc tag, auxin, gibberellin, steroids, proteins, hydrophilic organic compounds, and their analogues. For example, when the linker material is biotin, beads or a filter on which biotin-binding molecules such as avidin and streptavidin are bound to the surface can be used as the solid phase support. Similarly, when the linker material is glutathione, digoxigenin, digoxin, a His tag, a Flag tag, a Myc tag or the like, beads or a filter on which an antibody against these is bound to the surface can be used as the solid phase support.

The solid-liquid separation treatment is not particularly limited as long as it is a method capable of recovering the solid phase support in the solution in a state of being separated from the liquid component, and can be appropriately selected and used from among the known treatments used for the solid-liquid separation treatment. For example, when the solid phase support is particles such as beads, the solid phase support may be precipitated to remove the supernatant by allowing the suspension containing the solid phase support to stand or to be subjected to centrifugal separation, or the suspension containing the solid phase support may be filtered using filter paper or a filtration filter to recover the solid phase support remaining on the surface of the filter paper or the like. Further, when the solid phase support is a magnetic bead, it is possible to bring a magnet close to the container that contains the suspension containing the solid phase support, and to remove the supernatant after the solid phase support is converged on the surface of the container closest to the magnet. When the solid phase support is a membrane or a filter, the suspension containing the solid phase support is allowed to permeate through the solid phase support so that the complex containing both the β-1,3 glucan-binding molecule and the β-1,6 glucan-binding molecule is retained on the solid phase support and the free labeling material is separated and removed.

The solid phase support from which the free labeling material has been removed by the solid-liquid separation treatment may be directly used for detection of the complex containing both the β-1,3 glucan-binding molecule and the β-1,6 glucan-binding molecule, or may be subjected to a single or several washing treatments. Water or the above-mentioned buffer can be used for the washing treatment.

The method for detecting a complex containing both the β-1,3 glucan-binding molecule and the β-1,6 glucan-binding molecule is not particularly limited. The complex may be detected directly as it is by mass spectrometry or the like, or it can also be detected by using a signal emitted by a labeling material with which the β-1,3 glucan-binding molecule or the β-1,6 glucan-binding molecule has been labeled.

When the complex containing both the β-1,3 glucan-binding molecule and the β-1,6 glucan-binding molecule is detected using a fluorescence signal emitted by a fluorescent material with which the β-1,3 glucan-binding molecule or the β-1,6 glucan-binding molecule has been labeled as an indicator, it is sufficient as long as the fluorescence signal emitted by the fluorescent material derived from the complex can be detected. That is, the fluorescence signal emitted from the fluorescent material in the complex may be detected, or, after the fluorescent material is separated from the complex, the fluorescence signal emitted from the separated fluorescent material may be detected.

For the separation of the fluorescent material from the complex, only the fluorescent material may be separated from the complex, or the β-1,3 glucan-binding molecule or β-1,6 glucan-binding molecule labeled with the fluorescent material may be separated from the complex. The fluorescence signal emitted by the fluorescent material in the complex or the fluorescent material separated from the complex may be measured by, for example, a method of measuring the intensity of fluorescence emitted from all the fluorescent molecules in the solution, or a method of measuring fluorescence intensity for each molecule can also be used.

For example, either one of the β-1,3 glucan-binding molecule and the β-1,6 glucan-binding molecule is labeled with a fluorescent material in advance, and the other is labeled with a linker material for immobilization on the solid phase support, and after incubating a mixture of a test sample, the β-1,3 glucan-binding molecule and the β-1,6 glucan-binding molecule for a predetermined time as needed, the solid phase support is further added to the mixture, followed by incubation for a predetermined time if necessary. Then, the labeling material that is not bound to β-1,3-1,6-glucan is removed from the solid phase support, and after washing once or several times as necessary, the fluorescence intensity of the solid phase support, that is, the total intensity of fluorescence emitted from the fluorescent material contained in all the molecules immobilized on the solid phase support is measured. The fluorescence intensity of the fluorescent material contained in all the molecules immobilized on the solid phase support after removing the labeling material that is not bound to β-1,3-1,6-glucan may be measured by separating the fluorescent material or the molecule to which the fluorescent material is directly bound from the solid phase support.

The fluorescence intensity of the solid phase support can be measured by a conventional method using a fluorescence spectrophotometer such as a fluorescence plate reader. The fluorescence intensity of the solid phase support depends on the amount of fluorescent material in all the molecules immobilized on the solid phase support. Accordingly, for example, by performing the same measurement with respect to β-1,3-1,6-glucan having a known concentration in advance instead of the test sample, and creating a calibration curve showing the relationship between the concentration of β-1,3-1,6-glucan and the fluorescence intensity, the amount of the fluorescent material of the complex containing both the β-1,3 glucan-binding molecule and the β-1,6 glucan-binding molecule immobilized on the solid phase support, that is, the amount of β-1,3-1,6-glucan contained in the test sample can be quantified.

When the complex containing both the β-1,3 glucan-binding molecule and the β-1,6 glucan-binding molecule is not immobilized on a solid phase support, or is immobilized on a bead-like solid phase support such as magnetic beads which can be dispersed in a solvent, the complex can be suspended in a solvent. In this case, it is also possible to detect, and to quantify based on the detection result, each complex by measuring the fluorescence intensity for each molecule using the suspension of the complex as a measurement sample solution.

Examples of the method for measuring the fluorescence intensity for each molecule in the sample solution include fluorescence correlation spectroscopy (FCS) (see, for example, Japanese Unexamined Patent Application, First Publication No. 2005-098876), a fluorescence intensity distribution analysis (FIDA) method (see, for example, Japanese Patent No. 4023523), and a scanning single-molecule counting (SSMC) method (see, for example, Japanese Patent No. 05250152). In addition, the measurement may be performed using a single molecule detection scanning analyzer described in Published Japanese Translation No. 2011-508219 of the PCT International Publication, a fluorescence single particle detection device disclosed in Japanese Unexamined Patent Application, First Publication No. 2012-73032, or the like. Among them, in the present invention, the measurement is preferably performed by the SSMC method because the fluorescent material can be quantitatively detected from even a smaller amount of sample with high sensitivity.

It should be noted that FCS, FIDA, and SSMC can be carried out by a conventional method using, for example, a known single molecule fluorescence analysis system such as MF20 (manufactured by Olympus Corporation).

For example, by performing statistical analysis after detecting the fluctuation of the fluorescence intensity of the molecule existing in a focal region in a confocal optical system by FCS, the number of molecules of the fluorescent material derived from the complex containing both the β-1,3 glucan-binding molecule and the β-1,6 glucan-binding molecule in the measurement sample solution can be calculated.

Further, by performing statistical analysis after detecting the fluctuation of the fluorescence intensity of the molecule existing in a focal region in a confocal optical system by FIDA, the number of molecules of the fluorescent material derived from the complex containing both the β-1,3 glucan-binding molecule and the β-1,6 glucan-binding molecule in the measurement sample solution can be calculated.

Moreover, using an optical system of a confocal microscope or a multiphoton microscope, by detecting fluorescence from a light detection region while moving the position of the light detection region of the optical system in a solution by SSMC, the number of free molecules of the fluorescent material derived from the complex containing both the β-1,3 glucan-binding molecule and the β-1,6 glucan-binding molecule in the measurement sample solution can be calculated.

The number of molecules of the fluorescent material derived from the complex containing both the β-1,3 glucan-binding molecule and the β-1,6 glucan-binding molecule in the measurement sample solution determined by the SSMC method or the like reflects the number of molecules of β-1,3-1,6-glucan that has been contained in the test sample. The larger the amount of β-1,3-1,6-glucan contained in the test sample, the larger the number of molecules of the fluorescent material calculated by the SSMC method or the like. Accordingly, β-1,3-1,6-glucan can be quantified by calculating the number of molecules of the fluorescent material derived from the complex containing both the β-1,3 glucan-binding molecule and the β-1,6 glucan-binding molecule in the same manner using β-1,3-1,6-glucan having a known concentration as a test sample in advance, and creating a calibration curve showing the relationship between the amount of β-1,3-1,6-glucan and the calculated number of molecules of the fluorescent material.

When the amount of the fluorescent material derived from the complex containing both the β-1,3 glucan-binding molecule and the β-1,6 glucan-binding molecule is measured by using a fluorescence signal, the measured fluorescence signal may be used as it is as the amount of the fluorescent material, but if the measurement background level cannot be ignored, the amount obtained by subtracting the background is preferably used as the amount of the fluorescent material.

In addition, the complex containing both the β-1,3 glucan-binding molecule and the β-1,6 glucan-binding molecule can also be measured by an immunochromatography method, a dot plot method, a slot blotting method, and a measurement method using an antigen-antibody reaction such as an ELISA method. For example, when the immunochromatography method is used, an anti-β-1,3 glucan antibody is used as a β-1,3 glucan-binding molecule, and this is immobilized in advance at a predetermined position on a test strip for immunochromatography. In addition, the β-1,6 glucan-binding molecule is labeled in advance using an enzyme or the like for chemiluminescence as a labeling material. As the enzyme, an enzyme commonly used as a label such as an alkaline phosphatase (AP) and a horseradish peroxidase (HRP) can be used. A measurement sample solution is prepared by mixing the test sample and the enzyme-labeled β-1,6 glucan-binding molecule in a solvent such as a buffer, and after incubation for a predetermined time if necessary, the measurement sample solution is added dropwise onto a test strip for immunochromatography and diffused on the test strip by the capillary phenomenon. Then, a complex that contains the β-1,6 glucan-binding molecule and is formed by binding to the anti-β-1,3 glucan antibody immobilized on the strip is detected by chemiluminescence by an enzymatic reaction. It is also possible to use an anti-β-1,6 glucan antibody immobilized on a solid phase support in advance as the β-1,6 glucan-binding molecule, and to label the β-1,3 glucan-binding molecule with an enzyme or the like for chemiluminescence as a labeling material in the same manner.

It is also preferable to assemble the aforementioned β-1,3 glucan-binding molecule and the β-1,6 glucan-binding molecule used in the method for measuring β-1,3-1,6-glucan according to the present invention into a kit. The measurement method can be performed more easily by using this kit. In addition to the β-1,3 glucan-binding molecule and the β-1,6 glucan-binding molecule, this kit can also include various reagents, equipment and the like used in the measurement method. For example, in this kit, it is possible to further include a solid phase support, a buffer for preparing a reaction solution containing a β-1,3 glucan-binding molecule, a β-1,6 glucan-binding molecule and a test sample, an instruction for the measurement method and a method for using the reagents included in the kit, and the like.

β-1,3-1,6-glucan can be detected specifically by distinguishing it from β-1,3-glucan or β-1,3-1,4-glucan by the method for measuring β-1,3-1,6-glucan according to the present invention. For this reason, this method is effective for quantifying β-1,3-1,6-glucan in a sample that may contain β-1,3-glucan or β-1,3-1,4-glucan, and is particularly suitable for evaluating the likelihood of infection of animals with a fungus.

That is, the method for evaluating a likelihood of fungal infection according to the present invention includes: a step of performing the method for measuring β-1,3-1,6-glucan according to the present invention using a biological sample collected from a test animal as a test sample, and measuring the amount of β-1,3-1,6-glucan in the test sample; and a step of evaluating the likelihood of infection of this test animal with a fungus based on the obtained measured value, that is, the amount of β-1,3-1,6-glucan in the test sample obtained by the above measurement. In this evaluation method, since β-1,3-1,6-glucan can be detected separately from β-1,3-glucan and β-1,3-1,4-glucan, it is possible to prevent a sample containing plant-derived β-glucan from becoming a false positive.

The test animal to be evaluated in the method for evaluating the likelihood of fungal infection according to the present invention is not particularly limited as long as it is an animal that does not originally contain β-1,3-1,6-glucan, and may be a human or an animal other than humans. Examples of the test animal other than humans include domestic animals such as pigs, cattle, horses, sheep and goats, experimental animals such as mice, rats, rabbits and monkeys, and pet animals such as dogs and cats.

The larger the amount of β-1,3-1,6-glucan in the test sample, the more β-1,3-1,6-glucan derived from the fungus contained in the test sample. Accordingly, for example, a threshold value that serves as a reference for evaluating the likelihood of fungal infection can be set in advance. If the amount of β-1,3-1,6-glucan in the test sample is below a predetermined threshold value or below the detection limit, the animal from which the test sample has been collected is evaluated as unlikely to be infected with a fungus. On the other hand, if the amount of β-1,3-1,6-glucan in the test sample is equal to or more than a predetermined threshold value, the animal from which the test sample has been collected is evaluated as likely to be infected with a fungus.

The threshold value used to evaluate the likelihood of fungal infection can be set experimentally. For example, the method for measuring β-1,3-1,6-glucan according to the present invention is used for a group for which fungal infection has been confirmed by another test method in advance and a group for which fungal infection has not been confirmed, and a threshold value capable of distinguishing both groups can be set as appropriate by comparing the measured values of both groups.

In addition, fungal infection can be monitored by performing the method for measuring β-1,3-1,6-glucan according to the present invention on test materials collected over time from the same animal. For example, when the amount of β-1,3-1,6-glucan in the test sample collected from the animal at a certain point in time is higher than that of the same type of test sample before the collection of the test sample, the animal can be evaluated as likely to be infected with a fungus.

As a method for evaluating the likelihood of fungal infection by preventing the occurrence of false positives due to plant-derived β-glucan while utilizing the conventional test based on the Limulus reaction, for example, a method of carrying out, in addition to a step of detecting β-1,3-glucan by the Limulus reaction, a step of detecting β-1,3-1,4-glucan or β-1,4-glucan by utilizing the fact that the plant contains β-1,4-glucan as a constituent molecule can be mentioned. More specifically, when the amount of 13-1,3-glucan detected by the Limulus reaction is equal to or more than a predetermined threshold value, β-1,3-1,4-glucan or β-1,4-glucan is specifically detected by using an anti-β-1,4-glucan antibody or the like. It is expected that it will be possible to judge whether or not the β-glucan detected by the Limulus reaction is a plant-derived β-glucan by specifically detecting β-1,3-1,4-glucan or β-1,4-glucan. However, with this evaluation method, although it is possible to specify whether or not a sample to be measured contains β-glucan derived from a plant, it may cause false negatives when β-glucan derived from a fungus and β-glucan derived from a plant coexist. In addition, a step of detecting β-1,3-1,4-glucan or β-1,4-glucan must be added, which is complicated. On the other hand, the method for evaluating the likelihood of fungal infection according to the present invention is excellent because it is less likely to cause false negatives and does not require a step of detecting β-1,3-1,4-glucan or β-1,4-glucan, thereby decreasing the number of steps.

In addition, for example, a method that includes a step of removing β-1,3-1,4-glucan from a sample to be measured, prior to the step of detecting β-1,3-glucan by the Limulus reaction can also be mentioned. More specifically, β-1,3-1,4-glucan is removed from the sample by using an anti-β-1,4-glucan antibody or the like. By removing β-1,3-1,4-glucan in advance, β-1,3-glucan can be detected with no plant-derived β-glucan present in the sample. As a result, detection of fungus-derived β-1,3-glucan with higher accuracy is expected, as compared to the conventional detection of β-1,3-glucan by the Limulus reaction. However, in this evaluation method, a step of removing β-1,3-1,4-glucan must be added, which is complicated. On the other hand, the method for evaluating the likelihood of fungal infection according to the present invention is excellent because β-glucan derived from a fungus can be detected separately from β-glucan derived from a plant without removing the β-glucan derived from the plant in advance, thereby decreasing the number of steps.

### EXAMPLES

Next, the present invention will be described in more detail with reference to Examples and the like, but the present invention is not limited to the following Examples.

<Preparation of Candida albicans Beta-Glucan (CSBG)>

A Candida albicans soluble β-glucan (CSBG) used in the subsequent experiments was prepared as follows.

Candida albicans IFO 1385 cells (2 g) degreased and dried with acetone were suspended in a 0.1 M NaOH solution, and after adding NaClO thereto, an oxidation treatment was carried out at 4° C. for 24 hours. After the oxidation treatment, the precipitate was collected by centrifugation (12,000 rpm, 15 minutes). The collected precipitate was washed with ethanol and acetone and then dried to obtain NaClO-oxidized Candida cell wall beta-glucan (OX-CA), which was a particulate form of Candida β-glucan. Furthermore, OX-CA was suspended in DMSO, sonicated, and then centrifuged, thereby obtaining a supernatant from which CSBG was obtained.

### <Preparation of Aspergillus Glucan (ASBG)>

An Aspergillus spp. soluble β-glucan (ASBG) used in the subsequent experiments was prepared as follows.

Aspergillus spp. mycelium (2 g) degreased and dried with acetone were suspended in a 0.1 M NaOH solution, and after adding NaClO thereto, an oxidation treatment was carried out at 4° C. for 24 hours. After the oxidation treatment, the precipitate was collected by centrifugation (12,000 rpm, 15 minutes). The collected precipitate was washed with ethanol and acetone and then dried to obtain NaClO-oxidized Aspergillus cell wall glucan (OX-Asp), which was an insoluble Aspergillus glucan fraction. Furthermore, OX-Asp was suspended in 8M urea, autoclaved (121° C., 20 minutes), and centrifuged, thereby obtaining a supernatant from which ASBG was obtained.

<Preparation of pollen beta-glucan (Pollen BG)>

A soluble β-glucan derived from Japanese cedar pollen (Pollen BG) used in the subsequent experiments was prepared as follows.

5 g of Japanese cedar pollen (manufactured by FUJIFILM Wako Pure Chemical Corporation) was suspended in 1.0 L of a 0.1 M aqueous sodium hydrogen carbonate solution, mixed with a stirrer for 30 minutes (at room temperature), and then centrifuged at 4° C. at 6,500 g for 5 minutes to collect a supernatant. The collected supernatant was further centrifuged (8,000 g, 5 minutes) to collect a supernatant. The collected supernatant was filtered using a 0.20 μm PES membrane filter, and the filtrate was stored at 4° C. as a crude extract. The crude extract was passed through an S-BGRP-immobilizing HiTrap column (BGRP column, 1 mL gel) (manufactured by GE Healthcare), and after adsorption, the BGRP column was washed with PBS. The adsorbate was eluted with 5 mL of 0.03 M NaOH and neutralized by adding 0.1 M citrate buffer (pH 3) to the eluate. The neutralized eluate was dialyzed while exchanging the external dialysis solution with 1.0 L of purified water four times (dialysis membrane: Spectra/por RC dialysis tube MWC01000), and a non-dialyzable fraction was frozen at −80° C., followed by freeze drying to obtain Pollen BG.

### Example 1

Three types of β-glucans with different origins were detected using fluorescently modified S-BGRP as a β-1,3 glucan-binding molecule and an enzyme-inactivated mutant of β-1,6-glucanase modified with biotin as a β-1,6 glucan-binding molecule. As the enzyme-inactivated mutant of β-1,6-glucanase, a mutant in which the 321st glutamic acid of β-1,6-glucanase derived from Neurospora crassa had been replaced with alanine was used.

Various β-glucans, Alexa Fluor 647-modified S-BGRP and a biotin-modified β-1,6-glucanase enzyme inactivated mutant were added to a phosphate buffer (1×PBS, 1% BSA) so that their concentrations were arbitrary, 0.5 μg/mL and 0.1 μg/mL, respectively, and then the resulting mixture was allowed to react at 37° C. for 30 minutes with shaking (reaction solution volume: 100 μL). Next, 10 μg of magnetic beads coated with streptavidin (650-01, manufactured by Thermo Fisher Scientific) was added, and the reaction was carried out at 37° C. for 1 minute with shaking. Subsequently, using a magnet, the magnetic beads in each solution were washed 5 times with 100 μL of washing phosphate buffer (1×PBS, 0.1% Triton X-100). 20 μL of Tris buffer for elution (10 mM Tris-HCl, 0.1% SDS) was added to the washed magnetic beads, heated at 95° C. for 1 minute, and then the supernatant was collected in a state where the magnetic beads were collected by a magnet. The collected supernatant was measured by the scanning single-molecule counting method.

In the measurement, a single molecule fluorescence measuring device MF20 (manufactured by Olympus Corporation) equipped with an optical system of a confocal fluorescence microscope and a photon counting system was used as an optical analyzer, and time-series photon count data were acquired from the above supernatant. At that time, the excitation light was irradiated at 1.3 mW using a laser beam of 642 nm, and the detection light wavelength was set to 660 to 710 nm using a band pass filter. The moving speed of the position of a light detection region in the sample solution was set to 90 mm/sec, the BIN TIME was set to 10 μs, and the measurement time was set to 600 seconds. Moreover, the measurement was performed once for each sample. After measuring the light intensity, the optical signals detected in the time-series data were counted from the time-series photon count data acquired for each supernatant. In a smoothing operation by the moving average method of data, the number of data points to be averaged at one time was 11, and the moving average process was repeated 5 times. Further, in a fitting operation, the Gaussian function was fitted to the time series data by the least squares method, and the peak intensity (in the Gaussian function), the peak width (full width at half maximum), and the correlation coefficient were determined. Furthermore, in a peak determination process, only the peak signals satisfying the following conditions were determined to be optical signals derived from the detection target, while the peak signals that did not satisfy these conditions were ignored as noise and the number of signals determined to be the optical signals derived from the detection target was counted as the “number of peaks”.

Peak Determination Process Conditions:

20 μs<[peak width]<400 μs

[Peak intensity]>1 (photons/10 μs)

[Correlation coefficient]>0.90

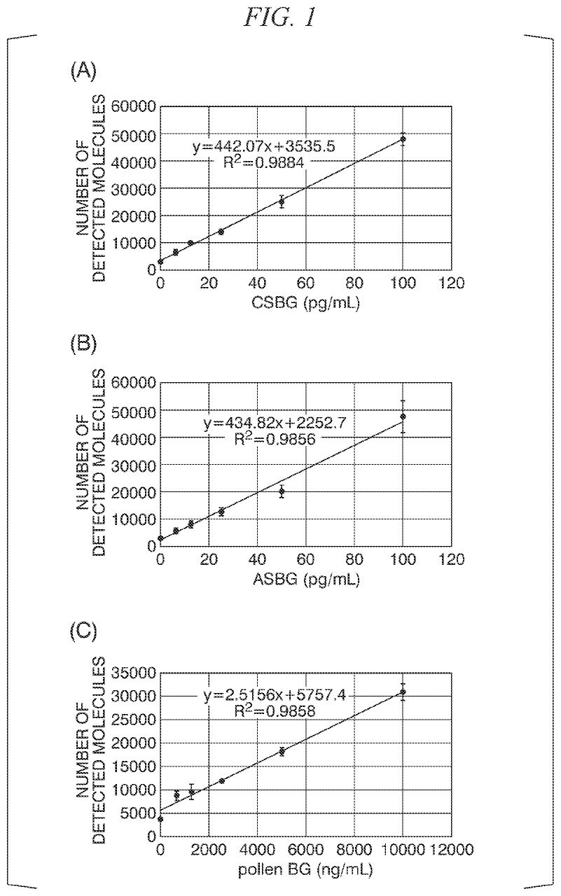

The measurement result using CSBG as a test sample is shown in FIG. 1 (A), the measurement result using ASBG as a test sample is shown in FIG. 1 (B), and the measurement result using Pollen BG as a test sample is shown in FIG. 1 (C), respectively. As a result, the detection limits of CSBG and ASBG were 3.4 μg/mL and 4.7 μg/mL at the final concentrations, respectively, demonstrating that the detection at very low concentration levels was possible. On the other hand, Pollen BG had a detection limit of 390 ng/mL at the final concentration, and the detectability was greatly reduced with respect to those of β-glucans derived from fungi. From these results, it was shown that it was possible to differentiate the β-glucans derived from fungi from the β-glucan derived from a plant to a high degree by a method of detecting a β-glucan forming a complex with both of the fluorescently modified S-BGRP and the biotin-modified β-1,6-glucanase E321 mutant.

### Reference Example 1

The three types of β-glucans used in Example 1 were detected by a test method using the conventional Limulus reaction. The test was carried out using Fungitec (registered trademark) G Test MK II (manufactured by Nissui Pharmaceutical Co., Ltd.). Table 1 shows the measurement results (Pachyman equivalents (reference material)) when the measurement was performed by preparing various β-glucans at 1,000 μg/mL.

TABLE 1

| | | Measurement |
|---|---|---|
| | | by Limulus |
| | | reaction |
| | Preparation | (pg/mL) |
| | concentration | Pachyman |
| β-glucan | (pg/mL) | equivalents |
| CSBG | 1,000 | 49.0 |
| ASBG | 1,000 | 81.9 |
| Pollen BG | 1,000 | 5.2 |

As shown in Table 1, in this test method using the Limulus reaction, it was confirmed that the measurement result of Pollen BG was about 1/10 of the measurement results of CSBG and ASBG, differentiation between the β-glucan derived from a fungus and the β-glucan derived from a plant was not sufficient, and false positives were likely to occur in samples containing the plant-derived β-glucan.

### Example 2

Three types of β-glucans were detected using the fluorescently modified S-BGRP and the biotin-modified β-1,6-glucanase E321 mutant derived from Neurospora crassa in the same manner as in Example 1 except that the fluorescence intensity was measured instead of the measurement by the scanning single-molecule counting method.

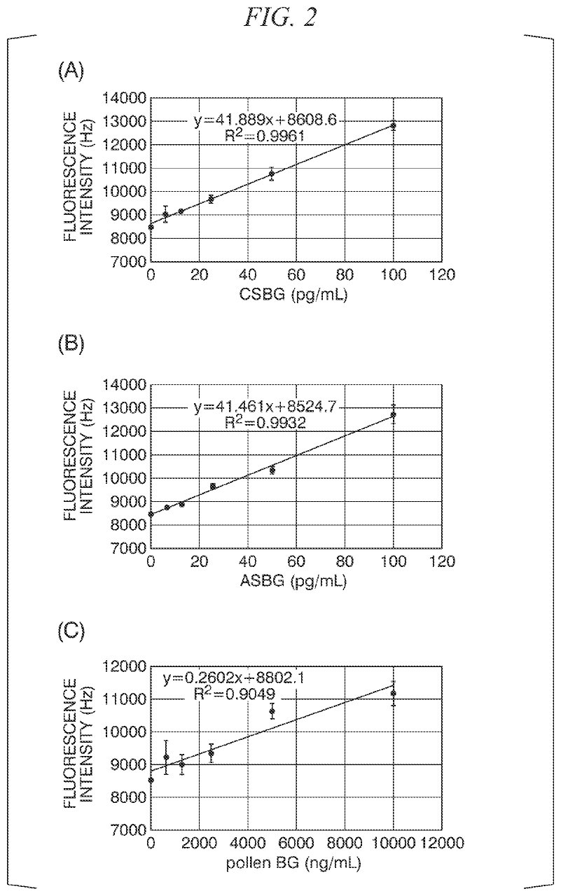

The measurement result using CSBG as a test sample is shown in FIG. 2 (A), the measurement result using ASBG as a test sample is shown in FIG. 2 (B), and the measurement result using Pollen BG as a test sample is shown in FIG. 2 (C), respectively. As a result, the detection limits of CSBG and ASBG were 11 μg/mL and 7.9 μg/mL at the final concentrations, respectively, demonstrating that the detection at very low concentration levels was possible. On the other hand, Pollen BG had a detection limit of 2,000 ng/mL at the final concentration, and the detectability was greatly reduced with respect to those of β-glucans derived from fungi. From these results, it was shown that it was possible to differentiate the β-glucans derived from fungi from the β-glucan derived from a plant to a high degree by a method of detecting β-glucan forming a complex with both of the fluorescently modified S-BGRP and the biotin-modified β-1,6-glucanase E321 mutant.

### Example 3

β-glucan contained in various immunoglobulin preparations was measured using the fluorescently modified S-BGRP and the biotin-modified β-1,6-glucanase E321 mutant derived from Neurospora crassa in the same manner as in Example 1. As the immunoglobulin preparations, Venilon (VENI) (manufactured by Teijin Pharma Ltd.), Venoglobulin 5% (VENO 5%) (manufactured by Japan Blood Products Organization), Gammagard (GAMM) (manufactured by Medley, Inc.), Glovenin (GLOV) (manufactured by Nihon Pharmaceutical Co., Ltd.) and Sanglopor (SANG) (manufactured by CSL Behring LLC) were used.

More specifically, β-glucan in each immunoglobulin preparation was measured in the same manner as in Example 1 except that 10 μL of the immunoglobulin preparation was added instead of β-glucan (reaction solution volume: 100 μL). Further, as a comparison target, the measurement by the conventional Limulus reaction was carried out in the same manner as in Reference Example 1. The results are shown in Table 2.

TABLE 2

| | Measurement | |
|---|---|---|
| | by Limulus | |
| | reaction | Method of |
| | (pg/mL) | Example 1 (pg/mL) |
| Immunoglobulin | Pachyman | CSBG equivalents |
| preparations | equivalents | (detection limit: 34 pg/mL) |
| VENI | ≤5.0 | <detection limit |
| VENO 5% | 48.4 | <detection limit |
| GAMM | 36.8 | <detection limit |
| GLOV | 73.6 | <detection limit |
| SANG | 1,570 | <detection limit |

As a result, β-glucan in each immunoglobulin preparation was not detected by the measurement method of Example 1. On the other hand, in the conventional measurement method based on the Limulus reaction, high concentrations of β-glucan were detected in some immunoglobulin preparations. It was assumed that this is because although these immunoglobulin preparations are contaminated with plant-derived β-glucan derived from the filter medium in the manufacturing process, and this plant-derived β-glucan (β-1,3-1,4-glucan) was detected in the Limulus reaction, in the method of Example 1, it was possible to specifically detect β-1,3-1,6-glucan so that the detection of plant-derived β-glucan was suppressed.

### Example 4

β-glucan was detected using biotin-modified S-BGRP as a β-1,3 glucan-binding molecule and an enzyme-inactivated mutant of fluorescently modified β-1,6-glucanase as a β-1,6 glucan-binding molecule. As the enzyme-inactivated mutant of β-1,6-glucanase, a β-1,6-glucanase E321 mutant derived from Neurospora crassa was used.

ASBG, an Alexa Fluor 647-modified β-1,6-glucanase enzyme-inactivated mutant and biotin-modified S-BGRP were added to a phosphate buffer (1×PBS, 1% BSA) so that their concentrations were arbitrary, 0.25 μg/mL and 0.25 μg/mL, respectively, and then the resulting mixture was allowed to react at 37° C. for 30 minutes with shaking (reaction solution volume: 30 μL). Next, 10 μg of magnetic beads coated with streptavidin (650-01, manufactured by Thermo Fisher Scientific) was added, and the reaction was carried out at 37° C. for 1 minute with shaking. Subsequently, using a magnet, the magnetic beads in each solution were washed 5 times with 100 μL of washing phosphate buffer (1×PBS, 0.1% Triton X-100). 30 μL of Tris buffer for elution (10 mM Tris-HCl, 0.1% SDS) was added to the washed magnetic beads, heated at 95° C. for 1 minute, and then the supernatant was collected in a state where the magnetic beads were collected by a magnet. The collected supernatant was measured by the scanning single-molecule counting method in the same manner as in Example 1. The measurement time was set to 600 seconds.

TABLE 3

| ASBG | Number |
|---|---|
| (ng/mL) | of peaks |
| 0 | 9,041 |
| 10 | 53,008 |

The measurement results are shown in Table 3. As shown in Table 3, the number of peaks increased depending on the concentration of ASBG, as in Example 1. From these results, it was shown that β-glucan derived from fungi can also be detected quantitatively, in the same manner as in Example 1, by a method in which, contrary to the method of Example 1, the β-1,3 glucan-binding molecule was modified with biotin and the β-1,6 glucan-binding molecule was fluorescently labeled.

### Example 5

By adding CSBG to human serum and calculating the additional recovery rate (%), the effect of human serum on the measurement of β-1,3-1,6-glucan was confirmed. The fluorescently modified S-BGRP and the biotin-modified β-1,6-glucanase E321 mutant derived from Neurospora crassa were used in the same manner as in Example 1. In addition, three types of human serum (manufactured by BioIVT LLC.) were used.

After adding 10 μL of human serum and 10 μL of 500 μg/mL CSBG to 60 μL of phosphate buffer (1×PBS, 1% BSA), the resulting mixture was incubated at 95° C. for 1 minute and then cooled in an ice bath. Subsequently, 10 μL of 5 μg/mL Alexa Fluor 647-modified S-BGRP and 10 μL of 1 μg/mL biotin-modified β-1,6-glucanase enzyme-inactivated mutant were added, respectively, and then the resulting mixture was allowed to react at 37° C. for 30 minutes with shaking (reaction solution volume: 30 μL). Next, 10 μg of magnetic beads coated with streptavidin (650-01, manufactured by Thermo Fisher Scientific) was added, and the reaction was carried out at 37° C. for 1 minute with shaking. Subsequently, using a magnet, the magnetic beads in each solution were washed 5 times with 100 μL of washing phosphate buffer (1×PBS, 0.1% Triton X-100). 20 μL of Tris buffer for elution (10 mM Tris-HCl, 0.1% SDS) was added to the washed magnetic beads, heated at 95° C. for 1 minute, and then the supernatant was collected in a state where the magnetic beads were collected by a magnet. The collected supernatant was measured by the scanning single-molecule counting method in the same manner as in Example 1. The measurement time was set to 600 seconds. As a control, the measurement result (number of peaks) when CSBG was added to a sample containing no human serum was taken as 100%, and the additional recovery rate (%) (=[Number of peaks of human serum-added sample]/[Number of peaks of human serum-free sample]×100) when CSBG was added to the same amount of serum was calculated.

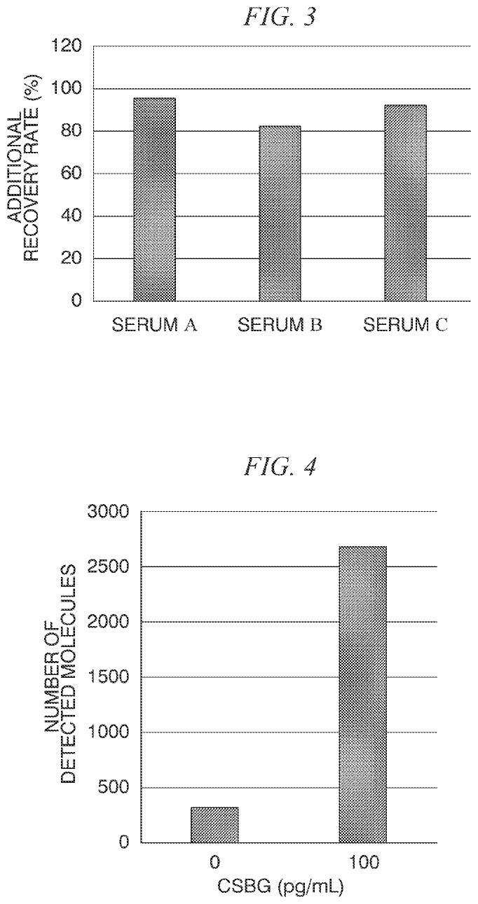

The measurement results are shown in FIG. 3. In all human sera, the additional recovery rate was around 90%. From this result, it became clear that the method for measuring β-1,3-1,6-glucan according to the present invention can be applied to clinical tests such as deep mycosis tests, since it can detect β-1,3-1,6-glucan in the serum, and can detect β-1,3-1,6-glucan derived from a fungus with high sensitivity.

### Example 6

CSBG was detected using fluorescently modified BmBGRP as a β-1,3 glucan-binding molecule and an enzyme-inactivated mutant of β-1,6-glucanase modified with biotin as a β-1,6 glucan-binding molecule. More specifically, CSBG was detected by performing the measurement by the scanning single-molecule counting method in the same manner as in Example 1 except that CSBG was used as β-glucan and Alexa Fluor 647-modified BmBGRP was used instead of Alexa Fluor 647-modified S-BGRP.

The measurement results are shown in FIG. 4. As shown in FIG. 4, it was possible to detect CSBG by using a combination of BmBGRP and the enzyme-inactivated mutant of β-1,6-glucanase. Therefore, it was shown that the β-1,3 glucan-binding molecule used in the present invention is not limited to S-BGRP shown in Example 1, and any of these β-1,3 glucan-binding molecules can be used.

### Example 7

CSBG was detected using fluorescently modified S-BGRP as a β-1,3 glucan-binding molecule and a biotin-modified anti-β-1,6 glucan antibody as a β-1,6 glucan-binding molecule. More specifically, CSBG was detected by performing the measurement by the scanning single-molecule counting method in the same manner as in Example 1 except that CSBG was used as β-glucan, a biotin-modified anti-β-1,6 glucan antibody was used instead of the enzyme-inactivated mutant of β-1,6-glucanase modified with biotin, and the anti-β-1,6 glucan antibody was added to a phosphate buffer (1×PBS, 1% BSA) so as to achieve a concentration of 1 μg/mL.

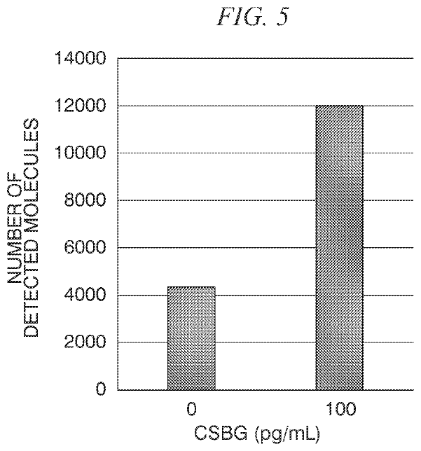

The measurement results are shown in FIG. 5. As shown in FIG. 5, it was possible to detect CSBG by using a combination of S-BGRP and the anti-β-1,6 glucan antibody. Therefore, it was shown that the β-1,6 glucan-binding molecule used in the present invention is not limited to the enzyme-inactivated mutant of β-1,6-glucanase shown in Example 1, and any of these β-1,6 glucan-binding molecules can be used.

### SEQUENCE LISTING

1. A method for measuring β-1,3-1,6-glucan,
the method comprising:
a step for mixing a β-glucan in a test sample, a molecule that specifically binds to a β-(1→3) bond, and a molecule that specifically binds to a β-(1→6) bond to form a complex containing said molecule that specifically binds to a β-(1→3) bond and said molecule that specifically binds to a β-(1→6) bond;
a step for detecting said complex separately from a β-glucan bonded to only one of said molecule that specifically binds to a β-(1→3) bond and said molecule that specifically binds to a β-(1→6); and
a step for measuring an amount of β-1,3-1,6-glucan in said test sample based on a result of said detection.

2. The method for measuring β-1,3-1,6-glucan according to claim 1,
wherein said molecule that specifically binds to a β-(1→6) bond is at least one selected from the group consisting of an enzyme-inactivated mutant of β-1,6-glucanase and an anti-β-1,6-glucan antibody.

3. The method for measuring β-1,3-1,6-glucan according to claim 1,
wherein said molecule that specifically binds to a β-(1→3) bond is at least one selected from the group consisting of a horseshoe crab-derived factor G or a mutant thereof, a protein containing a carbohydrate recognition domain of dectin-1 or a mutant thereof, a β-glucan recognition protein or a mutant thereof, an enzyme-inactivated mutant of β-1,3-glucanase and an anti-β-1,3-glucan antibody.

4. The method for measuring β-1,3-1,6-glucan according to claim 1,
wherein at least one of said molecule that specifically binds to a β-(1→3) bond and said molecule that specifically binds to a β-(1→6) bond is removed from said complex prior to the step for detecting said complex.

5. The method for measuring β-1,3-1,6-glucan according to claim 1,
wherein at least one of said molecule that specifically binds to a β-(1→3) bond and said molecule that specifically binds to a β-(1→6) bond is labeled with a labeling material, and
detection of said complex is carried out by detecting a signal emitted from said labeling material.

6. The method for measuring β-1,3-1,6-glucan according to claim 5,
wherein one of said molecule that specifically binds to a β-(1→3) bond and said molecule that specifically binds to a β-(1→6) bond is labeled with said labeling material, while the other is immobilized on a solid phase support.

7. The method for measuring β-1,3-1,6-glucan according to claim 6, wherein said solid phase support is a magnetic bead.

8. The method for measuring β-1,3-1,6-glucan according to claim 6,
wherein said solid phase support is modified with a biotin-binding molecule, and
among said molecule that specifically binds to a β-(1→3) bond and said molecule that specifically binds to a β-(1→6) bond, the molecule immobilized on said solid phase support is a biotin-modified molecule.

9. The method for measuring β-1,3-1,6-glucan according to claim 5, wherein said labeling material is a luminescent material.

10. The method for measuring β-1,3-1,6-glucan according to claim 9, wherein said labeling material is a fluorescent material.

11. The method for measuring β-1,3-1,6-glucan according to claim 1, wherein said complex is detected by a scanning single-molecule counting method.

12. A method for evaluating a likelihood of fungal infection,
the method comprising:
a step for performing the method for measuring β-1,3-1,6-glucan according to claim 1 using a biological sample collected from a test animal as a test sample to measure an amount of β-1,3-1,6-glucan in said test sample; and
a step for evaluating a likelihood of infection of said test animal with a fungus based on an amount of β-1,3-1,6-glucan in said test sample obtained by said measurement.

13. A kit for measuring β-1,3-1,6-glucan,
the kit comprising a molecule that specifically binds to a β-(1→3) bond and a molecule that specifically binds to a β-(1→6) bond, wherein
either one of said molecules is bindable to a solid phase support, and the other one binds to a labeling material which is detectable.


SEQUENCE LISTING (3 sequences)

SEQ ID NO: 1 - protein, 149 aa, Artificial Sequence
  Description of Artificial Sequence S-BGRP
MNHKVHHHHHHIEGRHMELGTYEVPDAKLEAIYPKGLRVSIPDDGFSLFAFHGKLNEEMEGLEAGTWSRDITKAKNGRWTFRDRNAELKIGDKIYFWTYVIKDGLGYRQDNGEWTVTGYVDEDGNPVDTDGPTTTPTGSEFKLVDLQSR

SEQ ID NO: 2 - protein, 480 aa, Neurospora crassa
  beta-1,6-glucanase
MYPPALTLLLTPGLVAAAIQPQAYASSADGRYKLSSYSAPVRGTGTPGSNSTWKLTIDDTPSGRKQTIKGFGAAVTDSTVSVFNALPSAQRTALLNTLMTTAGANFAMMRHTIASSDLSANPAYSYDDSNGQTDLSLSNFNLGGRGNAMASLLAEMRRLQPGLTILGSPWSPPGWMKLNRAIQGTTVNNNLDHAYASQFAQYFVKYLQAYQAKGATIDAITIQNEPLNSRAQMPTMYIYADEAGDLIQNNIGPALRNAGLDTKIWAYDHNTDQPSYPSTVLSRAGGYVPAVAWHCYASSLDWSVLTTFHNAHPGVEQYMTECWTSAKQPTPWNWAASFTMGPLQNWASGVTAWVLGTDTNDGPHLTGSDACDKCTGLVTVDAAAGTYNLRGDYYMMAQFSKFMKKGAVVMSGTGSWTYGDGSGLESVAATNADDGSRVVVIENKFGNEIYVTVEAKSGEVWSGLVYRNSVVTWVLPAAGA

SEQ ID NO: 3 - protein, 156 aa, Bombyx mori
  BmBGRP
MNHKVHHHHHHIEGRDDAKKDDAKKDDAKKDDAKKYEAPPATLEAIHPKGLRVSVPDEGFSLFAFHGKLNEEMEGLEAGHWSRDITKPKNGRWIFRDRNAALKIGDKIYFWTFVIKDGLGYRQDNGEWTVEGFVDEAGNPVNTEGSEITPGVEFSR